(19) United States
(12) Patent Application Publication
(10) Pub. No.: US 2012/0083666 A1
(43) Pub. Date: Apr. 5, 2012

(54) MEDICATION DELIVERY AND COMPLIANCE SYSTEM, METHOD AND APPARATUS

(76) Inventors: Donald Craig Waugh, Oakville (CA); Peter Gaspard Suma, Aurora (CA)

(21) Appl. No.: 13/080,466

(22) Filed: Apr. 5, 2011

Related U.S. Application Data
(60) Provisional application No. 61/320,772, filed on Apr. 5, 2010.

(51) Int. Cl. A61B 5/00; G06Q 50/22; G06F 17/00

(52) U.S. Cl. 600/300; 700/244; 705/2

(57) ABSTRACT

A system, method and apparatus for use in improving patient compliance with prescribed medication. The apparatus is configurable as a node on a communication network and has a storage unit for storing medication elements for a prescribed course of treatment. A delivery unit delivers the medication elements in a scheduled order corresponding to the course of treatment. A notification unit notifies a patient at the medication delivery apparatus that a next scheduled one of the medication elements should be consumed. A compliance unit issues to the network an indication that the next scheduled one of the medication elements has been consumed by the patient. If no notification is received by the compliance agent, remedial action is taken.

### CROSS REFERENCE TO RELATED PATENTS

This patent application is claiming priority under 35 USC §119 to a provisionally filed patent application entitled “Method and Apparatus for Monitoring Medication Dispensing and Compliance”, having a provisional filing date of Apr. 5, 2010, and a provisional Ser. No. of 61/320772.

### FIELD OF THE INVENTION

This invention relates to a medication delivery and compliance system, method and apparatus. If a patient needs to take multiple medications, there is a risk of complications arising from failure to comply with the prescribed course of treatment. Records kept by emergency wards and long term care facilities show that among seniors, the incidence of non-compliance is particularly high. Studies show that more than 40% of seniors regularly take at least 5 or more medications for various ailments and approximately two thirds of all elderly patients cannot properly manage their medications, accounting for almost 25% of hospital emergency visits.

### DESCRIPTION OF RELATED ART

Medication delivery units are known in which pills are stored in dedicated compartments with indications at the delivery units for a patient as to what pills are to be taken at what times.

### SUMMARY OF THE INVENTION

According to one aspect of the invention, there is provided medication treatment compliance apparatus for use in a medication compliance system, the medication treatment compliance apparatus configurable as a node on a communication network and comprising:

a storage unit for storing a plurality of medication elements together comprising a prescribed course of treatment;

a delivery unit for delivering the medication elements in a scheduled order corresponding to the course of treatment;

a notification unit for notifying a patient at the medication delivery apparatus that the patient should take a next scheduled one of the medication elements;

a compliance unit for issuing to the network an indication that the next scheduled one of the medication elements has been consumed by the patient.

The medication treatment compliance apparatus preferably forms one node on a communication network, the compliance unit operable to issue the notification to another node on the network for the attention of a compliance agent. The patient can actuate the compliance unit to issue said indication to the network. Alternatively, a detection means can detect whether the patient has consumed the next scheduled one of the medication elements and can be operable automatically to issue the alert if it is detected that the patient has consumed the medication element.

The medication treatment compliance apparatus can further include video conferencing elements for providing video conferencing between the medication treatment compliance apparatus and another node on the communication network.

A medication treatment compliance system including a compliance apparatus as previously described can include at the other node, a compliance agent to monitor the alert on one of a scheduled basis and an on-demand basis. The system can further comprise a summons functionality for connection to the network to enable a person at the medication treatment compliance apparatus to summon assistance from the network. The system can further comprise a summons functionality for connection to the network to enable a person at a remote node on the network to summon a person at the medication treatment compliance apparatus.

The medication treatment compliance system can further comprising control hardware and software for controlling operation of the medication treatment compliance apparatus, at least a part of the control hardware and software located at a node remote from the medication treatment compliance apparatus.

The apparatus can further comprise a pre-emptive functionality for prompting delivery from the delivery unit of a medication element at a time earlier than a scheduled time for a patient to consume the medication element if complying with the prescribed course of treatment. This allows the patient to take the medication element to a location remote from the medication treatment compliance apparatus, the system further comprising a mobile device having an alert capability to alert a patient at the remote location to consume the medication element at the scheduled time for the patient to consume the medication element if complying with the prescribed course of treatment. The mobile device can have a compliance function actuatable by the patient to send onto the network an indication that a scheduled medication element has been consumed.

The medication treatment compliance apparatus can further comprise a primary care test unit for subjecting a patient to a test related to a property of the patient's condition, and for measuring the property to derive an indication of whether the patient has consumed the medication element. The primary care test unit can be operable to measure the property following one of a single instance of scheduled consuming of a medication element and a period corresponding to multiple instances of scheduled consuming of medication elements.

According to another aspect of the invention, there is provided a method of medication treatment compliance comprising, at a medication treatment compliance apparatus configured as a node on a communication network:

storing a plurality of medication elements in a storage unit, the medication elements together comprising a course of treatment;

configuring a delivery unit for delivering the medication elements in a scheduled order corresponding to the course of treatment;

operating a notification unit for notifying a patient at the medication delivery apparatus that the patient should take a next scheduled one of the medication elements;

issuing to the network an indication whether the next scheduled one of the medication elements has been consumed by the patient.

The method can further comprise a remote compliance agent to monitor whether said indication is issued. The method can further comprise establishing video conferencing between the medication treatment compliance apparatus and the other node on the communication network to investigate if the indication fails to issue. The method can further comprise operating a summons functionality connected to the network to enable a person at the medication treatment compliance apparatus to summon assistance from the network. The method can further comprise operating a summons functionality connected to the network to enable a person at a remote node on the network to summon a person at the medication treatment compliance apparatus.

The method can further comprise operating a pre-emptive functionality at the medication treatment compliance apparatus to deliver from the delivery unit a container containing a medication element at a time earlier than a scheduled time for a patient to consume the medication element if complying with the prescribed course of treatment, the patient taking the pre-emptively delivered medication element to a remote location, and receiving an alert from the network on a mobile device to alert a patient at the remote location to consume the pre-emptively delivered medication element at said scheduled time.

The method can further comprise conducting a test on the patient to measure a property of the patient's condition, comparing whether the measure of the property is consistent with the patient having consumed the medication element, and issuing said alert if the measure of the property is not consistent with the patient having consumed the medication element. The method can further comprise escalating a level of the issued alert in response to communication between the medication treatment compliance apparatus and another node on the network showing that a medical element scheduled to be consumed still has not been consumed by a predetermined period after the issuing of the alert. The escalating of the level of the issued alert involves the establishment of communication on the network with at least one of a pharmacist, a doctor, and emergency services.

According to one aspect of the invention, there is provided a cartridge of linked containers, each container containing a prescribed medication element, the containers and the respective medication elements ordered to correspond to a scheduled prescribed course of such medication elements.

According to another aspect of the invention, there is provided a medication delivery unit comprising a housing, a cartridge of linked containers stored in the housing, each container containing a medication element, the elements together comprising a pre-packaged prescribed course of medication treatment, the course corresponding to the order of containers in the cartridge, a feed mechanism for feeding a lead one of the containers from the housing to an access zone, and a separation mechanism for separating the lead one of the containers from a next adjacent container.

The cartridge can comprise a roll of such containers with the roll configured to be unwound progressively to reveal and enable access to successive containers, the medication delivery unit further including a drive mechanism to unroll the cartridge roll and a feed mechanism to feed a free end of the roll to an exit slot. The cartridge can alternatively comprise the containers being folded in a zigzag, concertina-style arrangement, the feed mechanism comprising a corresponding feed mechanism to unfold and draw off a lead one of the containers from the cartridge.

Preferably, the cartridge includes a series of separation zones, each separation zone located between adjacent ones of the containers, where separation of one container from another adjacent container is facilitated by a weakened portion of container material, each zone, for example, having a line of perforations across the container material. The line of perforations can be crossed by a small cut through the container material whereby, once a container has been detached from the roll, it can readily be opened by pulling at the exposed end of the cut.

The containers can be formed from stock material and stocked with medication elements at essentially the same time. In one method, prescribed medication elements such as preset quantities of pills are accurately positioned at storage sites on a web of a container material such as polyethylene which is intermittently driven past a container manufacturing station. At this station, the web is folded around a previously positioned medication element and then heat sealed around that element at its free edges and at the site of a separation zones. The web is then advanced one container length to present the next part of the web to receive a further medication element and to be formed as the next container. Once the correct sequence of medication elements have been packed sufficient for the prescribed course of treatment and for the desired scheduled length of the course, the web is cut and a new prescribed course is inserted in a further sequence of filling and forming steps. For liquid medication, a preformed sachet filled with the required amount of the medication is dropped onto the web material and then made up into a medicine container as previously described with respect to pills. The containers can alternatively be configured in other convenient forms such as blister packs, the cartridges including for example weakened zones between containers to permit the linked containers to be stored in cartridge form.

For a roll cartridge, the patient unit can include a bay into which the cartridge is dropped, the bay and the cartridge preferably having matched dimensions and shape such that the cartridge automatically seats in a stable supported configuration in the bay with a free end of the roll accessible for leading to an exit zone. The patient unit feed mechanism can comprise a drive wheel and a lever means which interact to grip a part of the cartridge roll material therebetween, the feed mechanism operable to drive a free end of the cartridge roll having containers formed therein when the drive wheel is driven. The drive wheel can be configured and located to apply drive to one or both side edges of the free end of the cartridge or can be configured as a roller spanning substantially the full width of the free end of the cartridge and gripping the free end against an idler roller.

The unit preferably includes a separation mechanism to enable easy manual separation of the leading container from an adjacent container after the leading container has been fed out of the unit through, for example, a delivery slot. The separation mechanism can be integral with the feed mechanism: for example, in the drive roller arrangement, gripping pressure applied to the free end of the cartridge behind the lead container is maintained or increased so that when the lead container is pulled it is torn away from the next adjacent container. Alternatively, the separation mechanism is from the feed mechanism such as a knife or bar which is configured to move from a neutral to an applied position either to grip the leading edge of the cartridge to allow tearing of the lead container or to cut through the container material at the separation zone between the lead container and the next adjacent container.

While a slot is suited as an exit route for flat containers, other forms of exit aperture are adopted for containers of different shape. For example, for a series of containers linked in the form of a tube, a more rounded exit aperture is used. Similarly, while it is preferred that the leading container exits the patient unit to allow breaking or tearing of the leading container from an adjacent container, the leading container can alternatively be fed from the interior of the unit to a protected annex region where a gate is automatically or manually lifted to reveal the scheduled container and to allow it to be detached from the cartridge.

The cartridge can be housed in a compartment in the patient unit including a locking lid or panel which can be hingedly mounted to permit the compartment to be opened for removal and replacement of a cartridge when the containers of the current cartridge are close to being used up, or for other servicing. Preferably, mounted in the compartment is a light source to illuminate a region of each container as it arrives at an inspection zone, the region bearing a barcode or like identifying indicia. A reader for reading the barcode or indicia or a camera for obtaining an image of the bar code or indicia can also be mounted in the compartment. Operation of the light source and the reader or camera are synchronized to enable reading of the barcode or other indicia or to enable such an image to be obtained. In one example, the light source and camera operate to obtain an image of the barcode of the container immediately behind the leading container in the direction of advance of the free end of the cartridge roll. Container images are communicated to a reader for reading the barcode or deciphering the other indicia. Information represented by the bar code or other image indicia is then compared to a stored data relating bar code or other indicia to information defining a course of treatment, which information may be stored either locally at the patient unit, or remotely from the patient unit.

As an alternative to a cartridge having returnable element, the cartridge can be constructed with a view to being fully discarded once the medication elements have been used up. In one arrangement, a series of containers is formed as a tube with sections of tube wall forming outer walls of respective containers. Preferably, the tube is of circular cross section with the tube wall weakened as by perforations periodically along its length and with annular sections of the wall between adjacent sets of perforations forming annular boundaries of successive joined disc-shaped containers. Internally of the tube, each container can be defined by top and bottom webs extending between and sealed at the tube wall.

In use of this type of cartridge, the patient unit can have a storage bay in which the tube is lodged, and a tractor mechanism to draw the tube progressively through the bay towards a separation station as containers are delivered in accordance with the patient's scheduled course of treatment. The patient unit can include a separation mechanism to separate a leading disc container from the remaining part of the tube and to present the disc container to a delivery aperture such as a slot. The patient or care giver can then open up the delivered disc container to access medication contained within it. The outer face of the tube has barcode or other indicia arrayed along its length, each barcode or other indicia characterizing the medication contained within the container at that position along the tube. Within the storage bay, a barcode reader or camera is mounted to read the barcode or obtain an image of the indicia. The information is used to ensure validity of the particular container for which medication is about to be delivered. It will be appreciated that use of the tube cartridge obviates the requirement for a returnable element. The tube in cross sectional form can be square, rectangular or any convenient alternative shape.

Regardless of the particular type of cartridge, when the cartridge is close to being exhausted of containers for the particular course of medication, either at the patient unit or from a remote location, a monitoring unit can monitor the imminent exhaustion of containers and can alert the pharmacist location that a new cartridge should be prepared for a further course of medication. Preferably, replacement medication is dispensed under the direction of a pharmacist at a pharmacy, with cartridge preparation occurring at a cartridge preparation and dispatch facility. The replacement cartridge can be dispatched by mail or courier to the patient or care giver at the patient unit before full exhaustion of the original cartridge. Alternatively, a replacement cartridge can be picked up from a pharmacy following arrangements to have the cartridge sent from a packaging site to the pharmacy.

The patient unit can form one node in a communication network and can further include system elements for use in an audio visual communication link, with other nodes on the network having corresponding system elements for use in an audio visual communication link with the patient or care giver at the patient unit. The system elements at the patient unit can include a screen for showing an image or live video of a remote contact person such as a pharmacist, doctor, care giver or call centre operator and a camera for obtaining an image or live video of the patient and transmitting the image to the remote contact person. The system elements at the patient unit can further include a speaker for projecting speech from the remote contact person and a microphone for receiving speech from the patient. The patient unit can further include a visible and/or audible alarm such as a lamp or buzzer to indicate to a patient when the drug or other medication in a lead container is scheduled. The patient unit preferably also includes an input unit for actuation by the patient or care giver to register in the system that the prescribed medication has been taken. This information can be directed to a remote central service unit or to a dedicated remote contact person either for immediate or scheduled monitoring. Finally, the patient unit can include a press-for-assistance button or like actuator to permit the patient to establish audio visual communication with the contact person and an alarm to signal to the patient that audio visual communication is desired by the contact person. At least some of the control means for the audio visual communication link, the operation of the camera and light sources, the alarm, the actuator, etc. can be located at the patient unit, while other of such control means can be located at a remote node or otherwise distributed on the network. In addition, as an alternative to physically actuated buttons, keys and the like at the patient unit, the patient unit screen can be formed with soft keys.

As an alternative or addition to capability within the patient unit being used to establish the unit as a network node for data, alert and video access to and from the patient, pharmacist, care giver, doctor, etc, the patient unit can function interactively with a mobile electronic device such as a cell phone or smart phone device to provide connection and processing capability. In such an arrangement, the patient unit can have a pre-empt function to allow early presentation and availability of a container or series of containers of medication scheduled to be consumed at a later time. This has value to a patient who is leaving the site of the patient unit. In these circumstances, the patient can take a container containing medication to be consumed together with the supporting smart phone device. The smart phone device can be programmed to issue an alarm sound or other indicator when it is actually time for the delivered medication to be taken. The patient can then turn off the alarm, take the medication and signal confirmation at the smart phone, the confirmation being conveyed onto the network by appropriately configured smart phone software. When the patient returns home, he or she docks the smart phone at the patient unit. When the next medication is due, the smart phone alarm sounds and the same procedure is followed at the home site. It will be appreciated that in this embodiment, the patient unit, other than the docked smart phone, is essentially a mechanical device with all the intelligence for the patient unit, other than that located elsewhere on the network being furnished by the smart phone, whether docked at the patient unit or lodged at some other location. The smart phone device and the patient unit can alternatively share processing and connectivity features as appropriate.

According to another aspect of the invention, there is provided a method of preparing a medication cartridge for a prescribed course of treatment comprising examining a prescription for the course of treatment, preparing medication corresponding to the prescription, packaging the medication as an ordered plurality of containers in the medication cartridge, the order of the containers corresponding to an order in which the medication elements are to be taken in compliance with the prescribed course, and applying identifying indicia to each container corresponding to the nature of the medication element in the respective container.

According to a further aspect of the invention, there is provided a method of delivering a medication comprising packaging within a cartridge of linked containers elements of a prescribed course of medication treatment, the course corresponding to the order of containers in the cartridge, storing the cartridge in a housing of a patient unit, successively feeding a lead one of the containers from the housing to an access zone, and separating the lead one of the containers from a next adjacent container.

According to yet another aspect of the invention, there is provided a medication treatment compliance system comprising a medication delivery unit having a housing and a plurality of medication elements stored in the housing, the elements together comprising a prescribed course of medication treatment, the unit further including a delivery mechanism to present medication elements in a prescribed order at a patient unit exit zone, and an inspection unit to inspect a container containing a medication element to be dispensed, the patient unit forming one node of a network for communicating an output from the inspection unit to a remote node of the network, and processing means and memory on the network, the memory storing application instructions for processing the information from the inspection unit, for interpreting the information from the inspection unit to determine the nature and scheduled time for consumption of a medication element to be dispensed, for comparing the determined nature and scheduled time for consumption against prior stored data corresponding to the prescribed course of medication treatment, and for validating a medication element to be presented in response to the comparison showing a match.

According to another aspect of the invention, the patient unit has a response unit for measuring a physical change in the patient in response to the patient's scheduled consumption of medication. The response unit is coupled into the drug compliance system. In operation, the patient is required to subject him- or herself to a unit for monitoring the expected physical change. If the patient fails to undergo the physical test or if, upon undergoing the physical test, the response unit does not detect the expected physical change, an alert is issued over the network that non-compliance is suspected. The physical test depends on the nature of the expected physical change which depends in turn on the drug being taken. The test may be a two-part test including measuring a patient condition before the prescribed drug is to be taken and then measuring the change in patient condition after the prescribed drug has been taken. The patient unit can be operable to issue both a primary alert corresponding to an apparent failure by the patient or care giver to implement a medication delivery procedure, and a secondary alert corresponding to the absence of an expected physical change following a scheduled consumption of medication. This aspect is of value for patients who may be particularly forgetful or who may have become distracted during the medication consumption procedure.

### BRIEF DESCRIPTION OF THE DRAWINGS

For simplicity and clarity of illustration, elements illustrated in the following figures are not drawn to common scale. For example, the dimensions of some of the elements are exaggerated relative to other elements for clarity. Advantages, features and characteristics of the present invention, as well as methods, operation and functions of related elements of structure, and the combinations of parts and economies of manufacture, will become apparent upon consideration of the following description and claims with reference to the accompanying drawings, all of which form a part of the specification, wherein like reference numerals designate corresponding parts in the various figures, and wherein:

FIG. 1 is a side view of a medication cartridge according to one embodiment of the invention.

FIG. 2 is a side sectional view of a series of pouches forming part of the medication cartridge of FIG. 1.

FIG. 3 is a top view of the series of pouches of FIG. 2.

FIG. 4 is a side view of an alternative form of medication cartridge according to an embodiment of the invention.

FIG. 5 is a sectional view of a series of boxes forming part of a medication cartridge according to another embodiment of the invention.

FIG. 6 is a perspective view of a medication cartridge according to another embodiment of the invention.

FIG. 7 is a sectional view of part of the cartridge of FIG. 6.

FIG. 8 is a perspective view of a medication delivery apparatus according to an embodiment of the invention.

FIG. 9 shows a detail of a variant of the medication delivery apparatus of FIG. 8.

FIG. 10 is a perspective view of a separation mechanism shown removed from a medication delivery apparatus according to one embodiment of the invention.

FIG. 11 is a side view of the separation mechanism of FIG. 10.

FIG. 12 is a sectional view from the front on the line A-A of FIG. 11.

FIG. 13 shows a front view of the medication delivery apparatus of FIG. 8.

FIG. 14 is a sectional view on the line A-A of FIG. 13.

FIG. 15 is a sectional view on the line B-B of FIG. 13.

FIG. 16 is a schematic view showing a network forming part of a system according to an embodiment of the invention.

FIG. 17 is a top view showing a stage in the production of a cartridge of the form shown in FIG. 1

FIG. 17A is a top view showing a later stage in the production of a cartridge of the form shown in FIG. 1

FIG. 18 is a perspective view of a cartridge according to an embodiment of the invention.

FIG. 19 is a perspective view of the cartridge of FIG. 18 shown in exploded view.

FIG. 20 is a side sectional view of a medication delivery apparatus according to another embodiment of the invention showing a hinged lid thereof in closed condition.

FIG. 21 is a side sectional view corresponding to FIG. 20 but showing the hinged lid in an open condition.

FIG. 22 is a perspective view of the medication delivery apparatus of FIG. 20 showing a cartridge stored within the apparatus.

FIG. 23 is a side sectional view of the apparatus of FIG. 22.

FIG. 24 is a detail of the illustration of FIG. 22 showing a mounting means for a cartridge.

FIG. 25 is a schematic view illustrating several use cases that may occur in the course of implementing a medication validation and compliance method according to an embodiment of the invention.

### DETAILED DESCRIPTION OF THE INVENTION INCLUDING THE PRESENTLY PREFERRED EMBODIMENTS

If a patient has many ailments, he or she may be prescribed a complex of drugs. Not only may the drugs differ from one another, but it may be required that they are taken at different times of the day, at variable doses, and with different periods between consumption, etc. This can place complex demands on the patient which are especially burdensome if the patient has difficulty in looking after him- or herself. If the patient cannot fully comprehend complex instructions, there is a danger the patient may either take the wrong drug, or may take the right drug but at the wrong time.

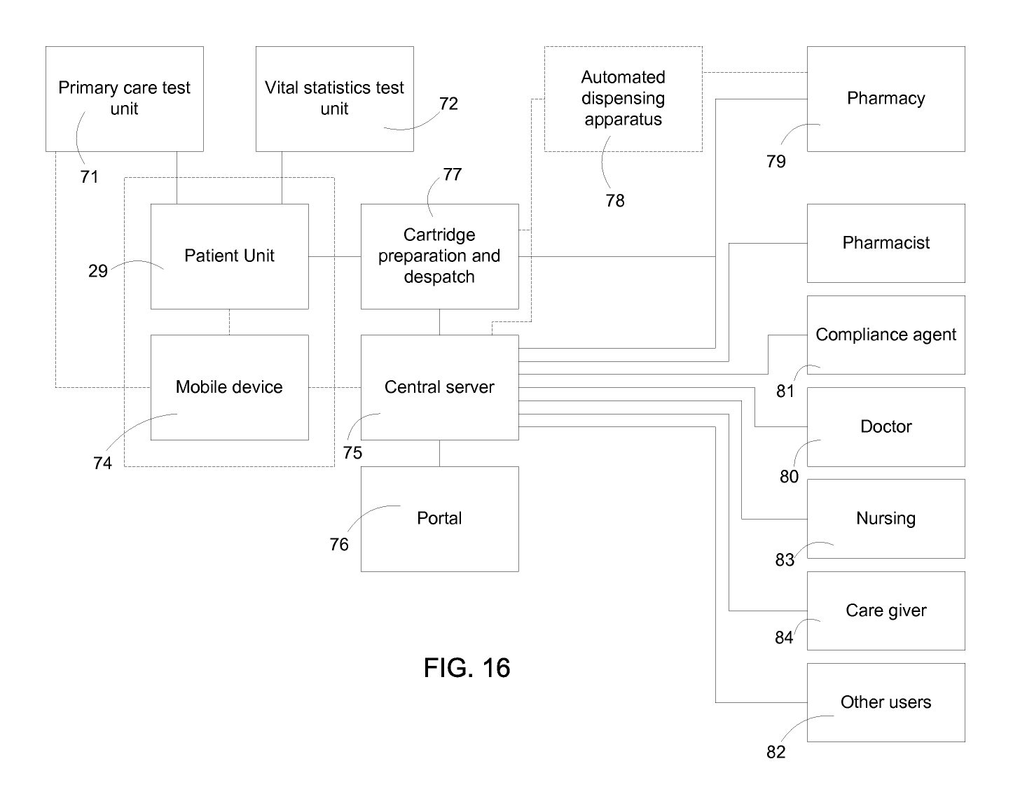

The apparatus, system and method of the invention offer a solution to the need for effective medication delivery and compliance, particularly for use in the home, but also for work sites, group homes, assisted living quarters, etc. The apparatus includes, in its simplest aspect, a patient unit and a medication cartridge, but has a more complex system aspect insofar as the patient unit forms a node on a communication network, one example of which is shown schematically in FIG. 16. The patient unit, medication cartridge and associated network functionalities to be described presently, provide a patient environment in which there is a reduced chance of confusion in relation to a patient's scheduled consumption of medicine, and in which non-compliance with a scheduled consumption is rapidly identified to enable appropriate action to be promptly taken.

The medication cartridge corresponds to a prescribed course of treatment for a patient at home or at another care facility. It is prepared at a pharmacy in compliance with a prescription issued by a physician and then either picked up from the pharmacy or shipped to the patient in readiness for use. A series of containers in the cartridge has an access order corresponding to an order in which the medication elements are to be taken in compliance with the prescribed course of treatment.

The patient unit is kept at the patient's home or other care facility, such as a work site, group home, assisted living quarters, etc. The unit has a housing in which the medication cartridge is stored and a medication delivery arrangement for delivering validated medication elements from the cartridge to the patient in the order and at the times that the medication elements are scheduled to be consumed by the patient. The patient unit 29 is connected into the communication network to ensure accuracy of delivery and to ensure compliance by the patient or assisting care giver with the prescribed course of treatment.

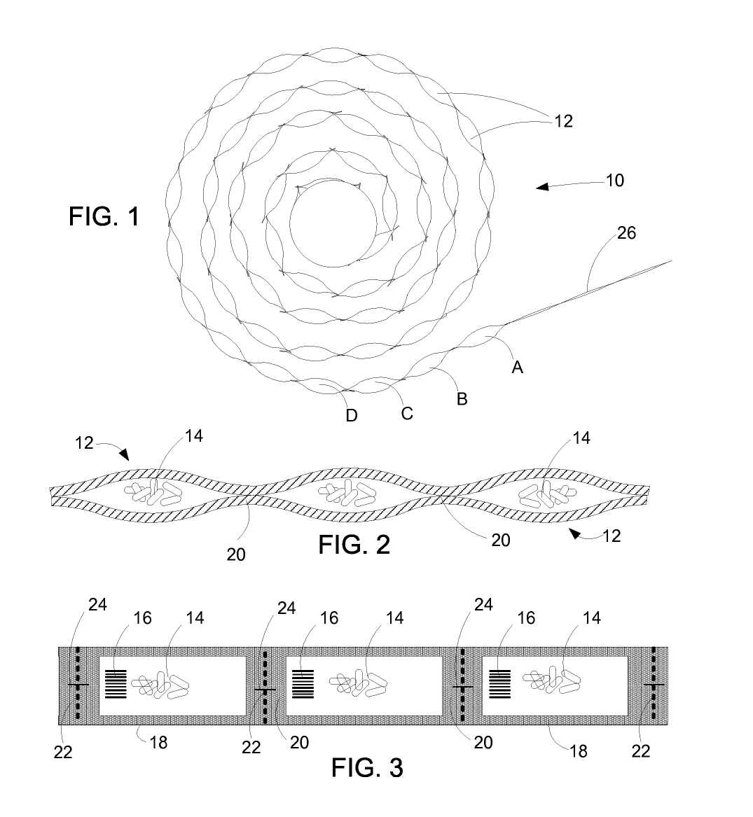

As shown in FIGS. 1 and 2, one embodiment of cartridge 10 has a series of linked containers 12, each containing a medication element 14. The containers are wound as a tight spiral on a reel, the order of containers in the cartridge, A, B, C, etc., corresponding to the scheduled order of a prescribed course of treatment for the patient, with the cartridge having been prepared by a pharmacist or pharmacy technician in compliance with the prescription. Each container may contain, for example, a single pill, lozenges, etc., a number of similar or dissimilar pills, or liquid preparations. As shown in the plan view of FIG. 3, each of the containers bears identifying indicia 16 such as a bar code which corresponds to the medication element in the respective container. The bar code may signify any or all of all of (a) the nature of the drug in the container, (b) the identity of the patient, (c) the day and time the particular dose in that container is to be taken, (d) the lot number and expiry date of the drug, (e) the size of the dose to be taken and other handling instructions, (f) the manufacturer, (g) the pharmacy site at which the course of medication was assembled and packed, (h) the pharmacist or pharmacy technician overseeing packing of the cartridge, (i) the cartridge serial number, (j) written physical description, etc.

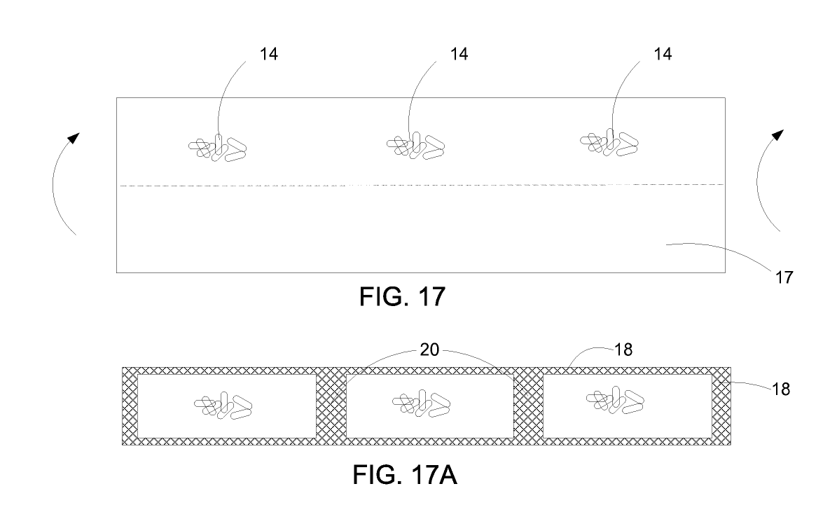

The roll of containers is configured to be unwound progressively to reveal and enable patient access to successive containers. As shown in FIGS. 17 and 17A, the roll is formed from a folded web 17 of thermoplastic material that is heat sealed along edge zones 18 and at separation zones 20 to create individual containers 12. Separation of one container from an adjacent container is facilitated by a line 22 of perforations across the container material which creates a weakened region. In this embodiment, each line of perforations is traversed by a small cut 24 through the sealed part of the container material whereby, once a container has been detached from the roll, it can readily be opened by pulling at the exposed end of the cut. As shown in FIG. 1, the cartridge roll 10 has a free end 26 comprising a series of containers that are empty of medication and are labeled with indicia identifying the patient for whom the cartridge is intended. The free end is used to facilitate cartridge loading and initial charging of a patient unit feed mechanism.

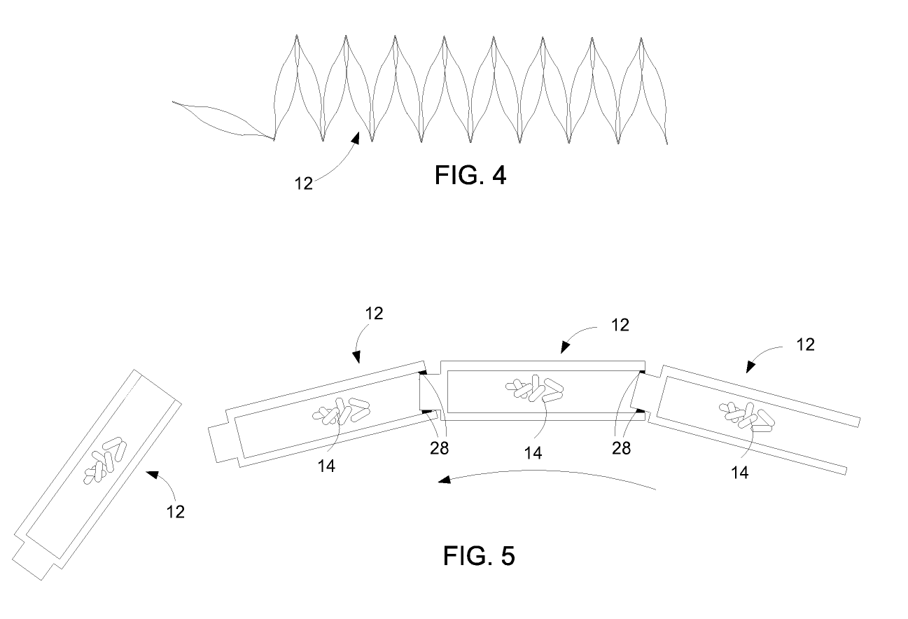

Various alternative designs of cartridge are possible. In one alternative embodiment as shown in FIG. 4, the cartridge containers 12 are folded in a zigzag, concertina-style arrangement. In another alternative, medication containers are made of stiffer, self-supporting material, so forming a less flexible container. In one embodiment, the cartridge 10 is formed as a series of flat, rectangular section, open-ended boxes as shown in FIG. 5. The cartridge forms a rectangular section tube having sufficient flexibility to be wound into a roll or drum and with a wall separating each container from an immediately adjacent container. In a container feeding operation, a foremost container is driven out of a delivery aperture in the patient unit as the drum is unwound. The exposed container is then broken away from its neighbour by stressing and breaking frangible link pieces 28, the act of breaking being such as to open up one end of the container that is broken away to enable access by the patient or care giver to the container contents.

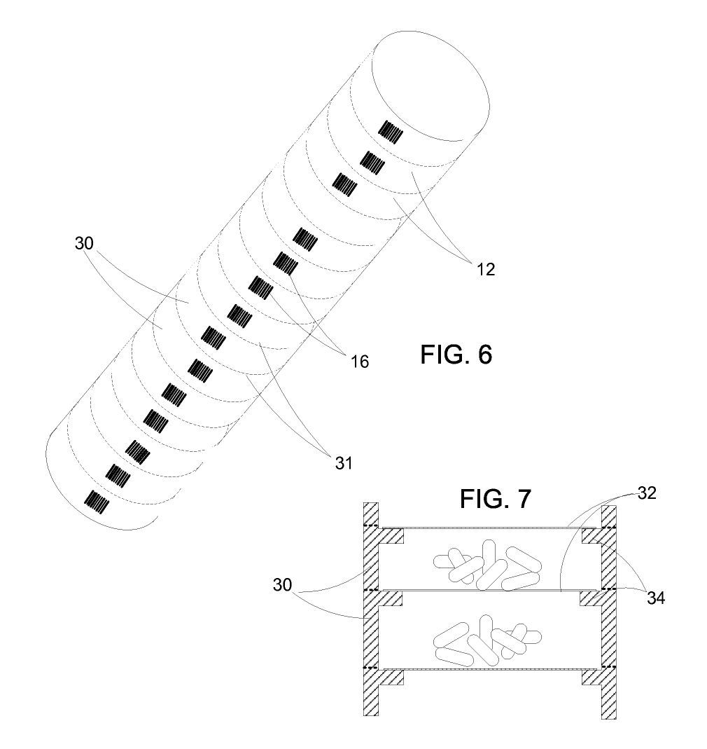

A further alternative type of cartridge is constructed with a view to its constituent elements being fully discarded once the medication elements have been used up. In the embodiment illustrated in FIGS. 6 and 7, a series of containers 12 is formed as a tube with a section 30 of tube wall forming an outer wall of a respective container. One form of tube is circular in cross-section with the tube wall weakened as by perforations 31 periodically along its length and with the annular section 30 of the wall between adjacent sets of perforations forming an annular boundary of a disc shaped container. Internally of the tube, each container is defined by a circular sealing element 32 adhering to an underlying annular ledge 34. The outer face of the tube has barcode indicia 16 arrayed along its length, each barcode characterizing the medication contained within the container at that position along the tube. Use of the tube cartridge does not require empty cartridge pieces to be returned to a pharmacy centre for rewinding or other recharging of packed medication. The tube in cross sectional form can be square, rectangular or any convenient alternative shape.

Containers used in all of the previously described embodiments of cartridge can contain pills, lozenges, liquid or other implementations of medication. The invention is valuable for liquid delivery because accurate delivery of small amounts of liquid medication is notoriously difficult. In the present case, the volume of the liquid dose is accurately metered at the pharmacy centre and provided there is no spillage upon subsequently opening of the container, an accurate volume is received by the patient.

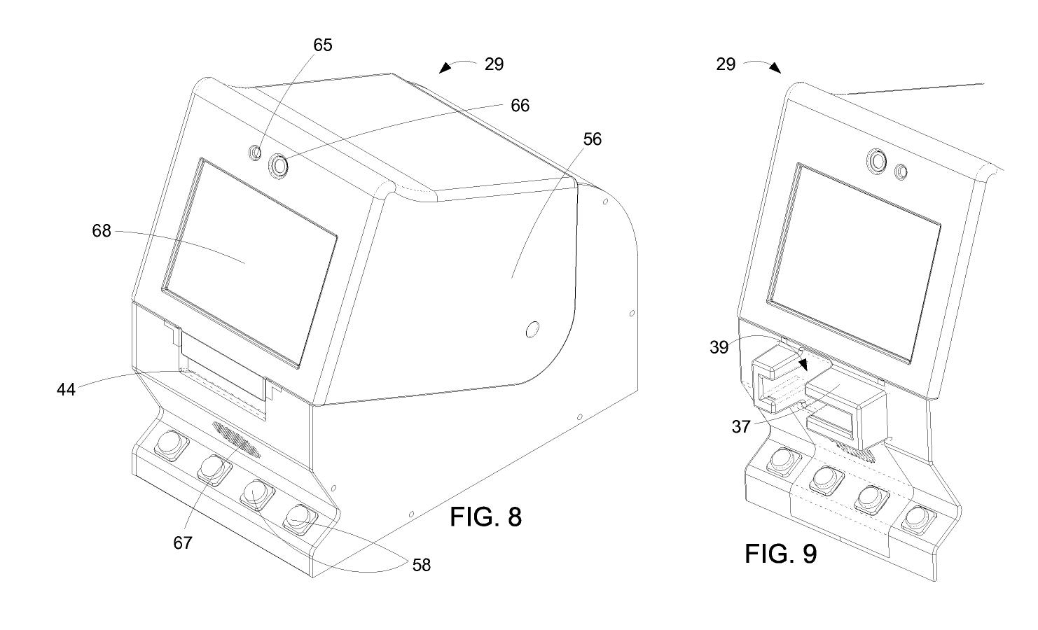

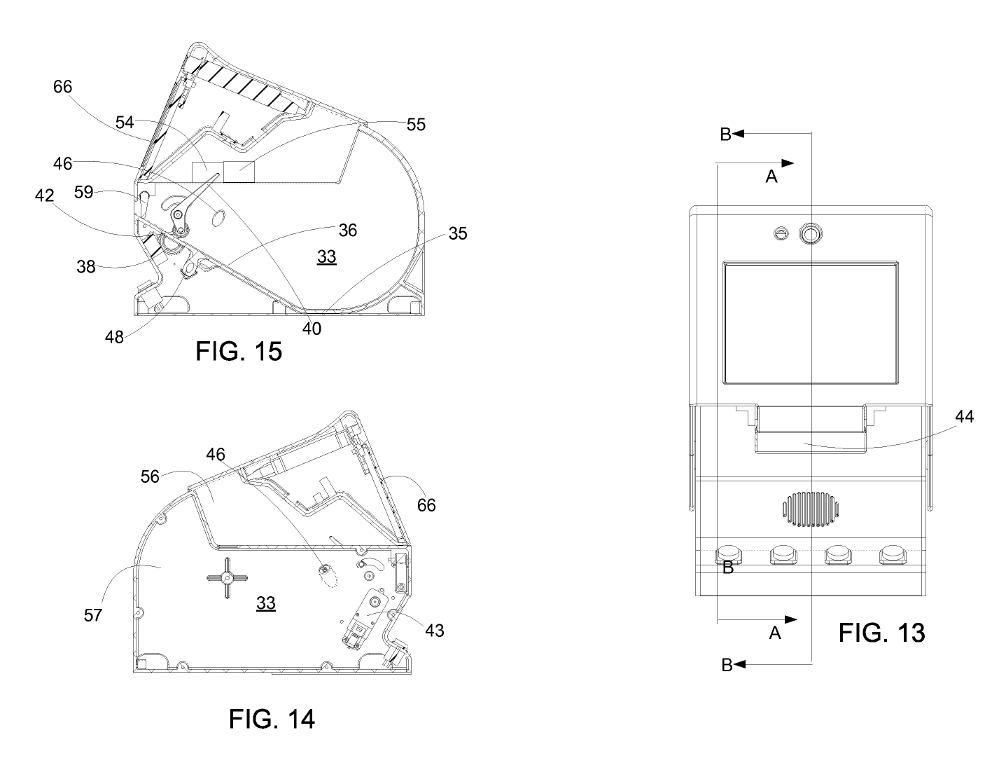

As previously mentioned, the delivery of medication from the different styles of cartridge 10 is effected using a patient unit 29 having a medication delivery mechanism matched to the particular cartridge design. A patient unit for the FIGS. 1-3 cartridge embodiment is shown in FIGS. 8-14. The patient unit has a housing 33, with the cartridge dimensioned to fit closely into the housing so that the cartridge roll seats against a floor 35 of the housing. Regardless of whether the cartridge is depleted by having containers progressively removed and delivered to the patient, the cartridge rolls down a ramp 36 to seat stably in the bottom of the housing.

The patient unit 29 has a feed mechanism which engages with the free end 26 of the cartridge roll. The free end 26 is located with its edge adjacent a drive wheel 38. When the roll is to be driven to eject a container for the scheduled administering of a prescribed drug, an over-centre spring lever 40 is actuated to drive an idler wheel 42 against the drive wheel and to pinch the edge of the cartridge roll free end between the wheels 38 and 42. Drive to the drive wheel is initiated from a motor 43 to drive the free end of the cartridge roll through a delivery slot 44. Drive to the wheel is halted when a sensor including an LED source 46 and a detector 48 show that the container immediately behind the ejected container has reached a predetermined position at which the separation zone 20, weakened by the perforation line 22, is under a grip bar. The grip bar is moved down onto the separation at a trailing part of the separation zone to grip the container material just behind the perforation line 22 to grip the roll against the ramp 36. This enables the patient or care giver to grip the ejected container which is suspended outside the delivery slot 44 and to tear the container away from the next adjacent container without further pulling the free end 26 through the slot.

An alternative form of exit slot 44 is shown in FIG. 9. The slot is surrounded by a wall 37 that projects from the patient unit fascia except at a cut-out 39. The wall 37 serves to constrain the user/patient to some extent from probing into the interior of the patient unit where moving elements of the feed mechanism are located. In addition, the cut-out 39 encourages the patient to grip a container 12 presented at the exit slot at a position spaced from the side edges of the container and in particular spaced from the container edge that is locked between the drive and idler wheels 38, 42. This gripping position for separating a container from the other containers of the cartridge is preferred as it tends to direct separation forces in such a way as to result in a clean separation of the container from the next-adjacent container in comparison with the container being gripped near one edge or being pulled sideways. It also reduces the chance of the containers being pulled away from the drive mechanism.

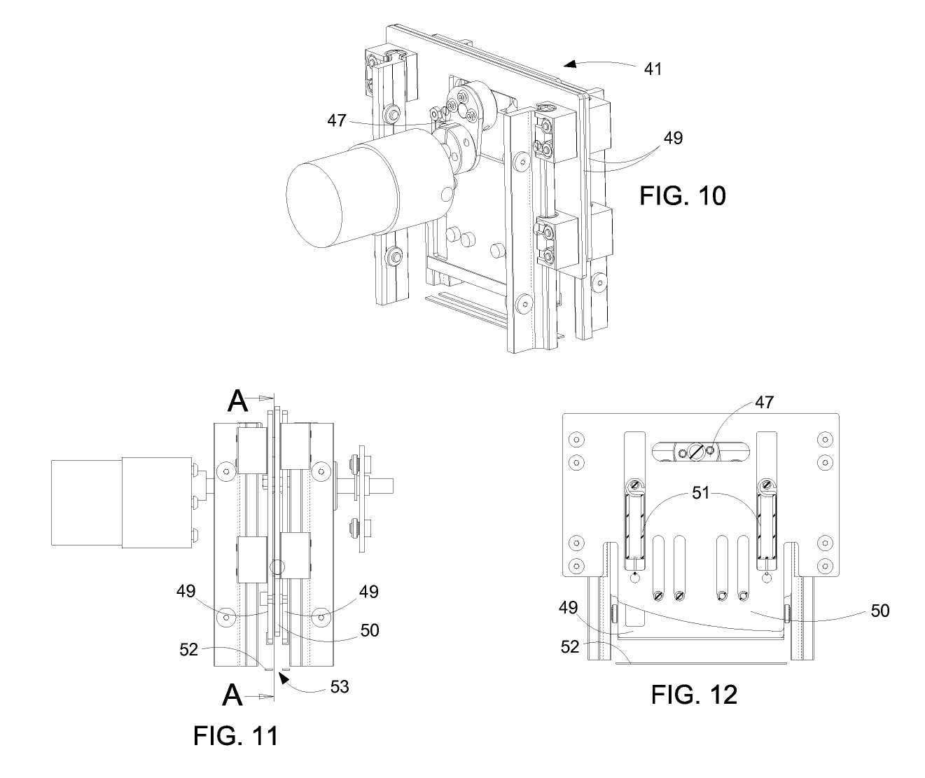

In separating the leading container from the rest of the cartridge roll, it is necessary first to clamp the free end of the roll at a position just behind the container to be separated, to effect separation, and then to remove the separated container from the patient unit. The separation and pulling can be separate movements or can be a common movement. Referring, to FIGS. 10 to 12, there is shown one form of separating mechanism, the mechanism being shown removed from its normal, installed position adjacent the exit slot 44. The separation mechanism has a trio of plates forming a sandwich structure shown generally as 41. The plates are mounted relative to the housing 33 to be moved as a group by operation of a motor 45 acting through a cam arrangement 47. The plate structure can be moved between a disengaged position and an engaged position. In the engaged position, the two outside plates 49 of the structure clamp the free end 26 of the roll at positions either side of a separation zone 20 between the container 12 to be separated and the next-adjacent container 12 of the roll cartridge 10. Once clamped, when the leading container is to be separated from the next adjacent container, the motor 45 is actuated to rotate a cam to drive a central plate 50 against the action of springs 51 to cut the web 17 in the manner of a guillotine at the separation zone. A base plate 52 has a slot 53 to accommodate the central plate 50 which is driven downwardly between the two outer plates 49 and past closely adjacent base plate edges defining the slot 53 so as to cut the web in a scissor action. In an alternative separation mechanism (not shown), the web is clamped by only one plate at a position rearwardly of the separation zone 20 between the containers 12 to be separated, with the leading container 12 then pulled by the patient or care giver to tear it from the rest of the web 17.

With the guillotine arrangement, if the patient is not present to effect separation of a scheduled container from the rest of the cartridge, the container may be ejected from the exit slot. For accommodating more than one container being ejected where a patient must consume medication elements from a series of containers at a single session, the ejected containers are collected in a collection unit such as a bucket or tray (not shown). The collection unit can be shaped and oriented to received, multiple containers 12 as a stack ready for the series of medication elements to be taken at some later time.

In an alternative embodiment of drive mechanism (not shown), one or both of the drive wheel and the idler wheel are configured as rollers to span the full width of the container material. If both rollers are long, the containers are gripped across their whole width although the gripping pressure must be limited to avoid damage to the contained medication elements. In an alternative, one roller is of full width so as to frictionally encourage the free end of the roll forward without however gripping it.

In the normal operation of the patient unit, when the leading container of the roll is to be delivered to the patient, the drive wheel 38 is actuated to drive the free end 26 forwardly to eject the foremost container through the exit or delivery slot 44. The engagement pressure between the rollers is set low enough not to damage the gripped container or its contents but high enough, firstly, that the engagement drives the cartridge roll forwardly and, secondly, to enable the foremost container to be torn away from the rest of the roll without further unwinding the roll. By appropriate control mechanisms, the pressure between the drive and driven rollers can be varied during the medication delivery procedure so as to optimize the pressure to the particular phase of the procedure.

Mounted in the upper wall of the housing 33 is a light source 55 to illuminate a barcode or other indicia 16 on the container 12 next in line to be delivered through the delivery slot 44. Also mounted in the housing is a reader or camera 54 to detect the bar code 16 or to scan an image of it and to feed corresponding bar code data or image data to an interpretation unit for deciphering the barcode or other indicia. The information decoded from the barcode or derived from the image is used to determine characterizing data such as the nature of the medication element contained in the container and its scheduled time for consumption. The housing 33 has a lid 56 which is hinged relative to a base 57 to enable opening of the patient unit to reveal the interior of the housing 33 into which a cartridge 10 is to be loaded or from which such a cartridge is to be unloaded and replaced. The camera/barcode reader 54 and the associated light source 55 are mounted in the hinged lid 56.

In a medication delivery operation, the drive wheel 38 grips one of the seamed edge zones 18 of the free end 26 of the cartridge roll against the idler wheel 42. The idler wheel is mounted at the end of the spring lever 40 which can be moved to an unengaged position to allow manual threading of the free end of the roll cartridge into a delivery bay immediately behind the exit slot 44. Once the roll free end is threaded, the lever arm 40 is moved to a bias position at which the wheels 38, 42 grip the free end 26 of the roll with the spring maintaining the idler wheel against the drive wheel pending manual release of the spring lever for subsequent cartridge replacement.

In use, if the roll cartridge 10 has not yet been opened and installed in the patient unit, it is opened and a free end 26 of the cartridge roll empty of medication is pulled off the roll. The housing 33 is opened by lifting the lid 56 and the cartridge 10 is dropped into the storage bay. The lid 56 is then lowered to close the unit.

A container containing a medication element may advanced to a staging position for inspection by the camera/barcode reader as part of a two movement process to deliver the container to the exit slot. Alternatively, the lead container may be advanced to the exit slot in the same movement that brings the next adjacent container to the inspection zone. The indicia on the staged container is inspected and characterizing data, such as the nature and scheduled time for consumption of the contained medication element, is determined. The determined characterizing data is validated against stored data corresponding to the patient's course of treatment and, in particular, against corresponding stored characterizing data for the next item scheduled to be consumed. If a match is found between the determined data and the stored data, the staged container is viewed as valid and, at the scheduled time for the patient to consume that medication element, he or she is alerted that his or her medication element is ready to be consumed. The match can be a combination of any of several elements of characterizing data viewed as suitable for establishing that an inspected container is valid and that the patient should be alerted that he or she should consume the presented medication element. Such characterizing data may include, for example, (a) data characterizing the nature of the medication element, (b) data characterizing a time of consumption, or period for consumption, of the medication element, (c) a confirmation that the current time of day is before an expiry data recorded in stored data for the medication element next scheduled to be consumed, (d) confirmation that the medication element is not the subject of any recall recorded in the stored data, etc.

If a valid output results from comparing the data determined by the inspection and the stored data, an alert is issued informing the patient that it is time to consume an available medication element. If no valid output is results, an appropriate warning alert is issued to the patient as a sound and/or light alarm and a corresponding signal is sent onto the communication network to inform a compliance agent at a remote node.

In one embodiment of the invention, the alert is advisory only: that is, the patient (or user, if the patient is being assisted at the patient unit) is informed of a valid or invalid output but notwithstanding the meaning of the alert message, the patient is free to consume the validated medication or not as he or she wishes. In another embodiment of the invention, if there is no match between the characterizing data on the medication container and the stored data for the medication next scheduled to be delivered, the patient unit delivers no further medication containers until the reason for the lack of validation has been investigated. As will be evident, the validation of product and the execution of a compliance function can be orientated very much towards patient control (and/or control by other human agents on the network such as a remote compliance agent).

Alternatively, control of the validation and compliance functions can be effected largely as automated process steps with the patient having little control over each process step and little freedom in the process outcomes.

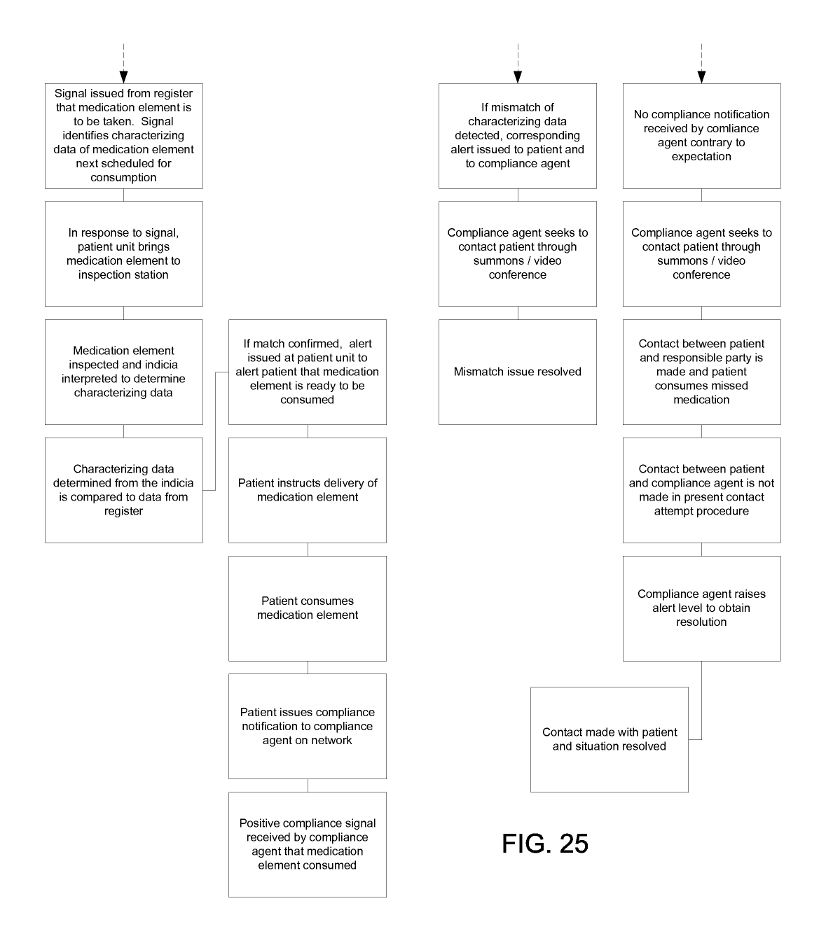

To illustrate operation of the patient unit, FIG. 25 illustrates three use cases. The left hand use case corresponds to a normal sequence of actions for validating medication, consuming it, and acknowledging that consumption has occurred. The other two are where a problem has occurred. It will be realized that for a combination of such actions, a large number of use cases can occur and that tailored responses will be implemented to deal with the implications of each sequence so as to resolve issues. This may call on various expertises in the network including a family member, another care-giver, a pharmacist, a physician, emergency services, data records repository for access to health, medication, or other data, a data records repository for storing records related to the sequence of events occurring at the patient unit, etc.

In a normal sequence, where the patient has received the alert that the medication element has been validated and so should be consumed, and the patient wishes to take the scheduled medication, he or she presses a designated key 58 to initiate the scheduled delivery procedure. This causes the spring lever 40 to pivot to bring the idler wheel 42 hard against the drive wheel 38 to grip the roll edge between the two wheels. A gate 59 at the delivery slot 44 is opened, and the drive wheel 38 is actuated to drive the free end 26 of the roll forwards so as to direct the previously validated container through the delivery slot 44. The roll is advanced one container length so that the next adjacent container is illuminated by the light source in the housing 33. The barcode 16 on the next adjacent container 12 is read by the barcode reader. The information signified by the bar code is compared with the stored data corresponding to the prescribed courses of treatment to ensure that the medication which is next to be delivered is consistent with the pharmacy record of the prescribed medication treatment and any other central records as necessary and appropriate. Meanwhile, the patient pulls the leading container 12 away from the delivery slot 44 and in so doing, tears it away from the next adjacent container 12. To prevent the patient's action from pulling more than the presented container from the roll at the scheduled medication delivery time, the gate 59 is mounted so that in its closed position, its bottom edge is lodged against the floor of the housing near the top of the ramp 36. As the roll is pulled, the contact between the roll and the gate 59 tends to drive the gate edge hard against the floor of the housing 33 and to lock the roll against further movement past the drive and idler rollers. In normal circumstances, the patient consumes the prescribed medication within a period set for consuming it as recommended by the pharmacist. Once consumed, the patient or care giver again presses the key 58 (or another dedicated compliance key) to indicate that the presented medication element has been consumed.

Timing of the opening and closing of the gate 59 and of the other elements of the feed mechanism is controlled by control logic, inputs to which include an input from an optical sensor having an emitter/detector combination 46/48. As the leading edge 26 of the roll is advanced towards the delivery slot 44, the sensor light passes through two layers of the container material together with an intermediate air gap in which the medication element 14 is located as it passes through the contents area of a container. Subsequently, as the roll material is advanced, the sensor light passes solely through the separation zone 20 where two layers of polyethylene have been heat sealed together. At the heat seal, light transmission is considerably higher than at the air gap region of the container, and the transition between the adjacent low and high light transmission areas is detected and used to govern operation of the feed mechanism and to determine the final position of the leading end of the cartridge roll.

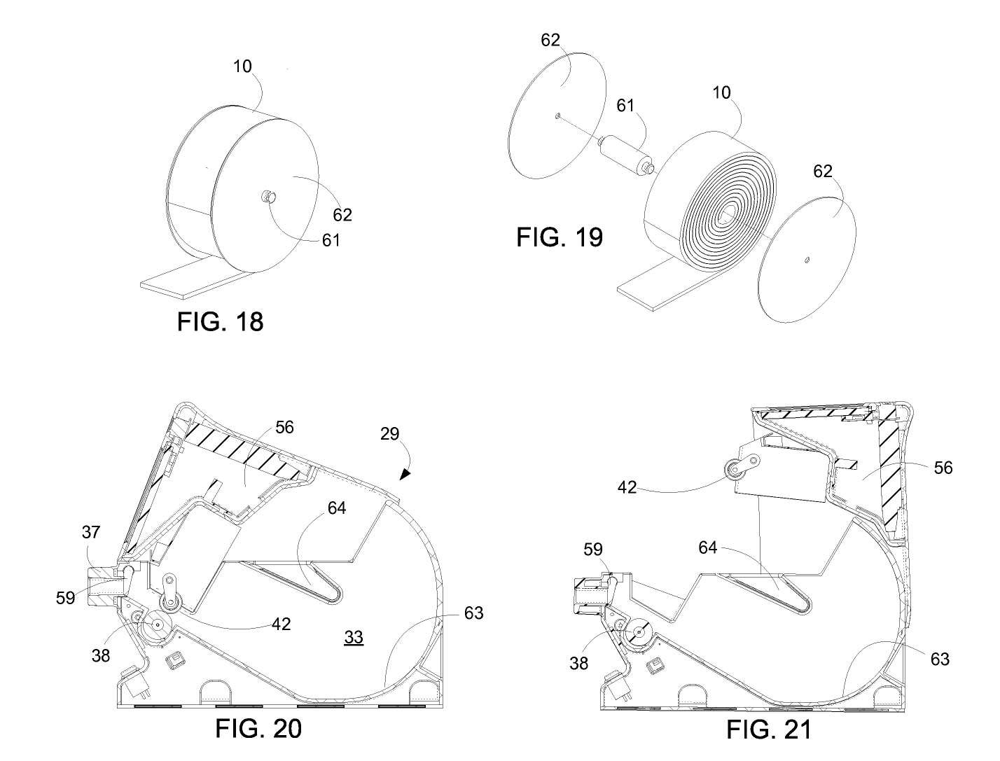

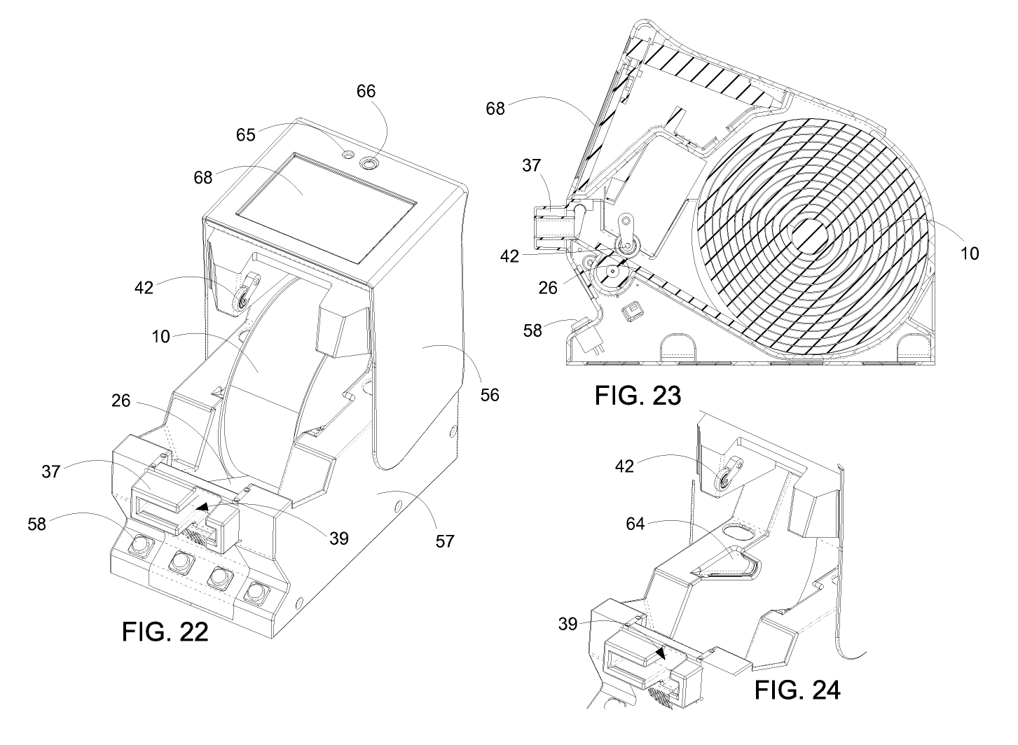

An alternative form of patient unit is shown in side sectional views in FIG. 20 (lid 56 closed) and FIG. 21 (lid 56 open). This patient unit is particularly adapted for a roll cartridge 10 of the form shown in FIGS. 18 and 19. The cartridge has a central axial member 61 linking confining plates 62 that are spaced by a distance marginally more than the width of the containers 12 with the container roll wound around the axial member 61. When the cartridge 10 is placed in storage bay 63 as shown in FIGS. 22 and 23, the axial member 61 auto-locates in a recess 64 in the housing wall as illustrated more clearly in the scrap perspective view of FIG. 24. The arrangement can include a lock to ensure that the cartridge 10 can only be dislodged by a positive unlocking action. The patient unit of FIGS. 20-24 has a modified cartridge drive arrangement in which the idler wheel 42 is mounted on the patient unit lid 56. In operation, after the cartridge 10 is inserted, the lid 56 is closed by moving it from the position shown in FIG. 21 to the position shown in FIG. 20. The action of closing brings the idler wheel against the drive wheel 38 with a gripping pressure sufficient to grip the container edges for driving the free end 26 of the linked containers. The required pressure is applied either by the weight of the lid 56 or upon a positive locking of the lid 56 into a closed position. As a further alternative to the FIG. 18 cartridge, the roll of containers is confined within a shell which includes raised bosses to function in the manner of the axial member 61 in supporting the roll in the patient unit housing. On installation in the patient unit, the free end of the roll is guided out of the shell through a slot aperture. When the cartridge is totally consumed, the shell is simply discarded.

The patient unit 29 incorporates an audio or audiovideo conferencing means including a microphone 65 and a camera 66 respectively for receiving the patient's or a care giver's speech and image and conveying them to a remote node on the communication network. The audiovideo conferencing means also includes a speaker 67 and screen 68 to enable a patient or care giver at the site of the patient unit to listen and watch a person at the remote node who may be a relative, a pharmacist, a doctor, a technician or other user. The particular expertise may be connect directly to the patient unit or initial communication may be between the patient unit and a call centre with call centre operators deciding on the particular expertise to assist the patient, The patient unit 29 includes other system elements as required for implementing the videoconference call. The patient unit screen 68 alternatively incorporates programmable touch keys and can be mounted either with a fixed orientation or so as to enable tilting to provide comfort to a patient or care giver who may be sitting or standing at the patent unit. The patient unit includes adjustment capability for enabling the patient to adjust the volume of speaker 67 and to alter the brightness/contrast of the screen 68.

When the patient is scheduled to take a dose of medicine, an audible and visible indicator at the patient unit 29 is triggered to apprise the patient. There may also be a text message displayed at the screen 68 or an audio message projected from the speaker 67, which message may be personalized to the patient to apprise him or her of the medication delivery event. In normal operation, the patient acknowledges that he or she wishes to proceed with the scheduled procedure, such as by pressing one of a number of keys whereupon the leading container, if previously validated, is separated from the remaining cartridge roll and exits the patient unit 29 at the delivery slot 44. The patient then removes and consumes the prescribed medication and acknowledges doing so by pressing a further one of keys 69. If no acknowledgement is provided by the patient within a certain period from the scheduled time scheduled time of consumption of the associated medication element, an alert is automatically sent from the patient site to family members, care providers, or anyone so designated to receive these alerts, and a further reminder as text or audio is presented at the patient unit 29 for the benefit of the patient, to be repeated periodically until the patient gives a required acknowledgement. The nature of the alert, its timing, the recipient of the alert signal on the communication network, and the response can all vary depending on the situation. The patient unit includes a capability for the patient or care giver to update any of the contact settings for any alert generated that is to be issued from the patient unit or to be transmitted from the network to the patient unit. A record of the settings may be stored locally at the patient unit or at any other node on the network accessible to the patient unit.

To minimize the chance of proper medication element verification and compliance being interrupted by loss of mains power, a backup battery is used to power light/sound indication alert apprise the patient that the patient unit may not be functioning sufficiently to give future reminders or present medication pouches to the patient.

The patient unit also offers a comfort link through the video link capability. For example, if the patient believes he or she is having a reaction from the medication, he or she can contact the call centre where an operator establishes dialog with the patient or care giver, makes a preliminary assessment of the patient's concern and may forward the call from the patient to an appropriate medical professional; for example, a pharmacist or doctor who may be the patient's regular professional if available or an alternative on-call professional if the patient's regular pharmacist or doctor is not available. In recognition of the cost, there may be an escalation of professionals depending on the emerging seriousness of the patient's situation. In addition, the expertise offered in the interface can be free or can be associated with a pay service, the network including a pay provision whereby the patient or care giver is informed of the cost of obtaining a desired consultation and expertise in order for the patient to decide whether he or she wants to accept the charge and proceed with the remote consultation.

The patient units can be built in various sizes depending on their required functionality. For example, the cartridges 10 can be relatively small to provide medication delivery for a short period of time of the order of a week, or can be larger to provide medication delivery of a complex mix of drugs requiring a larger number of containers per day or to provide medication delivery of a regular course of drugs for a longer period, of the order of a month. The housing can be any of a range of sizes to accommodate the different sizes of cartridge. In addition, a wider housing is used to mount two or more cartridges if medication delivery is to take place to more than one patient. In a further alternative embodiment, the patient unit is adapted to accommodate cartridges having differently sized containers that may originate, for example, from different manufacturers or from pharmacies with different standards. Such variably sized containers can be accommodated at a single patient unit by having bias means to operate on one or both of the cartridge, when loading, and on leading ones of the containers in the free end 26 when delivering containers to the delivery slot. The bias means can, for example, drive the cartridge to a register position at which the edge of the containers will be gripped when the drive and idler wheels are driven together. A further bias means such as one or more motor-driven or spring biased drive plates (not shown) may be used to drive individual containers 12 so as to supplement the cartridge registration.

Patient units of the type described above are located on a communication network together with other patient units and other nodes. At the other nodes are sited any or all of the functionalities illustrated in FIG. 16. Of particular significance are nodes which are involved in remote verification of medication to be delivered to a patient at the patient units and which permit remote monitoring of patient compliance in following a course of medication treatment.

In a further embodiment of the invention, the patient unit 29 has a network connection to an automated dispensing apparatus 78 of the sort described in copending U.S. Published Patent Application 20100198401 and U.S. Published Patent Application 20100268380. The automated dispensing apparatus 78 is itself connected on the network to a pharmacy 79 where a pharmacist has full purview and control of operations at the automated dispensing apparatus 78 and can authorize and enable dispensing of medication to a patient presenting a prescription at the dispensing apparatus. In a further embodiment of the invention, an automated dispensing apparatus of this type configured to dispense and package medication elements as a cartridge suitable for use in a patient unit. The cartridge corresponds to an ordered course of treatment. The patient's scheduled course of treatment is recorded on the network and/or can be recorded on the cartridge 10 at the time that the medication is being packaged. Subsequently, the cartridge is taken to the patient unit 29 and inserted into a storage bay serviced by a delivery unit to enable scheduled delivery of medication elements to a patient. The bay may be of any of the types described previously to accommodate a corresponding form of cartridge prepared at the automated dispensing apparatus 78 or the arrangement may use a design of cartridge which better lends to being prepared in an automated dispensing apparatus where space not given over to medication storage is limited and must be efficiently used.

An advantage of the patient unit 29 is that it is relatively portable and can be taken with the patient if the patient is to be away from his or her residence for an extended period. Set up at a new site is relatively easy requiring merely an Ethernet, USB, land line, cellular or wireless connection at the new site for establishing Internet connection to provide access to the compliance monitoring function and to enable access to records which may be stored at any of a number of nodes on the network.

The alternative cartridge designs have corresponding patient units configured for the particular form of cartridge. For the zigzag concertina style cartridge of FIG. 4, a feed mechanism operates to unfold and draw off a lead one of the containers from the cartridge in the course of a medication delivery procedure. For the cartridge of FIGS. 6 and 7, the patient unit has a storage bay in which the tube is lodged and a tractor mechanism to draw the tube through the bay towards a separation station. At the separation station, a separation mechanism separates the disc container from the remaining part of the tube cartridge and presents the disc container to a delivery bay such as a slot. The patient or care giver then opens up the disc container to access the medication contained within it. Within the storage bay is a barcode reader to read the barcode and, as described with respect to the prior illustrated embodiments, to ensure validity of the particular container for which medication is about to be delivered. As in the prior embodiments, when the cartridge is close to being exhausted of containers for the particular course of medication, a signal is sent from the patient unit to alert the pharmacist location that a new cartridge should be prepared for a further course of medication. This is dispatched to the patient or care giver before full exhaustion of the original cartridge.

For each of the different patient unit designs, the patient is required to press an indicator button or key 58 to report that medicine has been delivered and that he or she has consumed it. As an alternative to push button/keys, the patient unit screen 66 can have touch sensitive, programmable keys.

If no acknowledgement has been given by the patient or care giver within a certain period from the scheduled time for the medication to be taken, an alert is sent from the patient site and a further reminder as text or audio is presented to the patient at the patient unit, this being repeated periodically until the patient gives the required acknowledgement or until the situation must be escalated based on a preset assessment of risk. The nature of the alert, its timing, the recipient(s) of the alert signal on the network and the response can all vary depending on the situation. One important distinction in handling an alert is patient risk classification which depends, for example, on the seriousness of the patient missing a scheduled medication or the expected rate of deterioration of the patient's condition resulting from a failure to take the prescribed medication. In the case of a low risk patient, a failure to take scheduled medication may not be a serious matter and so apart from periodic reminders to the patient and a cautionary email automatically generated to a care giver nothing more is done until perhaps the third scheduled medication event in a row has been missed. At this point, an escalation takes place and the doctor is informed of the situation. This can prompt the doctor to initiate an audio or video link to the patient unit or between the doctor and care giver to identify why there have been several misses. If establishing a video link is not possible or the outcome is unsatisfactory, then a further escalation takes place with care giver or paramedic going to the patient site to ascertain the problem. In the case of a high risk patient, an alert may be sent within a short period of time of the omission to the care giver and the patient's doctor. At the same time, a call centre may seek to make contact regularly over a period of time after which paramedics are dispatched to the patient site.

Fundamental to the use of the patient unit as a network node is the idea of individuals, local or remote, who can act as compliance agents to monitor a patient's compliance with a prescribed course of treatment. Compliance information is accumulated and stored so as to be accessible by any of a number of authorized individuals in their capacity as compliance agent or in following up a situation identified by a compliance agent as problematic. At the first level is a care giver who may not be resident with the patient but who has responsibility for the patient. The care giver periodically accesses the website to get information about the compliance failure. The care giver's review of the situation may be triggered by a real time alert that there is a failure or the alert may be logged within the system ready for the next time the caretaker signs. The communication network includes the capability for the care giver to place an audiovideo call to the patient unit to follow up a compliance failure. Other levels of access can be provided to the patient's physician and pharmacist. Alerts signaling a patient's non-compliance are sent on a hierarchical basis, for example, if both the patient and primary care giver appear to have allowed a scheduled medication to pass without corrective action being taken within a prescribed time interval. Alerts signaling a patient's compliance may also be sent to authorized individuals as specified by the patient.

As shown in FIG. 16, the patient unit 29 is one of many on the network and is connected to a central server which functions to capture, store, analyze, modify and deliver data on the network, all in a secure manner. Information related to product validation and patient compliance is collected and can be relayed to any or all of a number of nodes including expertise such as a relative, a care giver 84, nursing 83, a doctor 80, a pharmacist 79, etc. Other information can be obtained from the network and, as necessary, delivered to the patient at the patient unit or to other users. Information related to validation and compliance for a single or multiple patient users can be made available at portals 76 where the selection, analysis and presentation of information is tailored to the requirements of particular users. Connections between the various functions depicted in FIG. 16 can be of any suitable means including hard wired and wireless. The patient unit 29 is one node on a communication network.

By means of the camera 66 at the patient unit 29, the server can provide remote monitoring of the patient in the manner of closed circuit TV to assess the patient's condition if something in the patient's handling of medications suggests that monitoring the patient's appearance or behaviour is warranted. The patient can, for example, be monitored by a nurse who periodically assesses the patient situation or assesses the patient situation in an event driven way such as when a medication is to be taken or when the patient acknowledges that he or she has taken medication.

The audio video link can also be used to download data and video related to the patient's activity. For example, if the prescribed course of treatment includes the patient starting to take an asthma medicine to be administered by an inhaler, the video link can be activated to apprise the patient by text, audio or video that it is known that the patient is to begin using an inhaler, and to enquire of the patient or care giver whether the patient would like to receive information and instruction related to the nature and operation of the inhaler. If the patient responds with an expression of interest, the information and instruction is made available, for example, as a text message or audio or video download.

In one embodiment of the invention, connection to the network is provided through the patient's or care giver's residential internet connection. Control to the patient unit is provided with a system on a chip device such as a RM7, RM9, Freescale IMX 2, these devices having communications means to access wireless and wired internet, control USB traffic, etc, and means to control serial devices such as cameras and displays.

The patient unit is managed from a remote monitoring call centre, the elements of the control including any or all of:
registering an alert if the patient or care giver at the patient site omits to take action required to initiate delivery of a prescribed medication within a predetermined period of the scheduled time for consuming the medication;
establishing audio visual connection from the centre to the patient/care giver site as and when required;
automatically or manually effecting a communication link from centre to the patient's doctor, hospital or pharmacist in response to any appropriate condition registered at the patient unit;
obtaining visual access at the centre to the interior of the patient unit to assess whether the failure to take a scheduled medicine arises from a fault of unit itself: e.g. jamming of the cartridge, problem with the feed mechanism, etc.;
automatically or manually effecting communication from the centre to a technician required for patient unit repair in response to any appropriate condition registered at the patient unit;
automatically or manually effecting communication from technician's site to the patient/care giver site to provide initial response to faulty patient unit—either the technician directs the patient or care giver to take corrective action or the technician can remotely actuate rudimentary corrective functionality in the patient unit, aided if necessary by some visual access of the interior of the patient unit by means of the installed light source and camera;
automatically or manually reporting on the status of any or all aspects of the electronic and mechanical components of the patient unit; and
with patient consent, obtaining temporary access to medication reminders and compliance event data for the purpose of troubleshooting operational problems of the patient unit.

The system response to a particular situation can have automatic elements to avoid demands on professional time that may not be needed in the circumstances but to involve the active participation of professionals when required. To minimize costs, there can also be an escalation of professional involvement at appropriate occurrences, the escalation typically moving through care giver, pharmacy technician, pharmacist and physician. Profiles are stored on the network for each patient to connect situations and corresponding responses for the patient. The profiles may include appropriate escalation and may govern what is done automatically, when someone is to become directly involved in the situation, and who that person or persons should be. Clearly the time frame both for response and escalation is made much shorter for a high risk classified patient than a low risk classified patient.

A primary source of value of the medication delivery apparatus described arises from its presence as a node on a communication network. As previously described, connection from the medication delivery apparatus to another node or nodes on the network may be used in the course of verifying that medication about to be delivered to a patient or care giver at the delivery apparatus is the right medication in terms of implementing a prescribed course of treatment. In addition, connection to another node or nodes may be used in the course of the notification being issued that a medication scheduled as part of a course of treatment has in fact been consumed by the patient for which it is prescribed.

As previously described the medication delivery apparatus has video conferencing functionality and this may be used to allow a patient interaction to be extended beyond the verification and notification functions described. For example, the video conferencing facility can be used to establish calls with pharmacists, doctors, nurses and care-givers. Some of this may involve charge billing activity.

The access to the network means also that records associated with the verification and compliance methods described can be posted to the network and stored at another node. This might for example be part of a store of the patient's medication and health record or can be part of statistical records. Similarly, the patient is able to access data from remote nodes that may be germane to verification and compliance in the course of a patient following a course of medication, or may be for a different purpose entirely. While access to the network in the course of the verification and compliance methods described will normally be at specific times related to a patient's scheduled and actual consumption of medication, functionalities and expertise on the network can be made available to the medication delivery apparatus at any time.

It will be appreciated that the verification and compliance method and apparatus of the invention is of particular value for patients who may have poor memory and/or who may have attention difficulties in terms of following a complicated pill consumption that may be relatively complex in terms of both the nature of medication elements to be taken in compliance with a course of treatment and the scheduled times for such consumption. As previously indicated, the schedule for a prescribed course is stored either locally or at a remote location on the network and is used in the course of establishing verification and compliance. If the schedule data is stored locally, it can, for example, be applied to the cartridge at the time the cartridge is prepared. The schedule can be made available at any time on the patient unit screen at the request of the patient or in the context of the patient communication with other on the network so that the patient can check scheduled consumption times and the medication to be consumed at such times.

The network access capability of the patient unit can also be used to browse through a directory of care providers and health professionals as a prelude to initiating audio-video communications with them with a view to setting up a service relationship.

In addition to the patient unit being a network node for data, alert and video access to and from patient, pharmacist, care giver, doctor, etc., the patient unit in a further embodiment, as illustrated in FIG. 16, interacts with a mobile device 74 such as a cell phone or smart phone having both computing and network connection capabilities. The mobile device 74, when connected to the patient unit 29, is used to control any or all of the patient unit functions including delivering medication elements and triggering compliance alarm and communication. In its use as a mobile device, a patient who may be someone who is about to travel to work or another site, actuates a pre-emptive function at the patient unit to obtain early delivery of the next-scheduled medication container. The patient takes the container with him or her together with the smart phone device. The smart phone is programmed such that when it is time for the scheduled medication to be taken, the smart phone sounds an alarm. The patient turns off the alarm, takes the medication and signals compliance confirmation at the smart phone, the confirmation being conveyed onto the network by appropriately configured smart phone software. When the patient returns home, he or she docks the mobile device at the patient unit. When the next medication is due to be taken, the alarm is triggered by a signal from the mobile device and the same procedure is followed at the home site. Alternatively, the patient records at the patient unit a specific return date and time when pre-empting presentation of medications for future consumption periods. The patient unit automatically takes over the medication consumption reminders at the return date and time, removing the necessity for docking the mobile device at the patient unit upon returning to the patient unit locale. It will be appreciated that in this embodiment, the patient unit is essentially a mechanical adjunct device, and it is the mobile device that fulfils most of the functions of the patient unit. All the intelligence for the patient unit is furnished by the mobile device, whether docked at the patient unit or present at some other location. Whether computing and networking capability is present only in the patient unit, only at the mobile device, or is distributed between the two, updating is effected from the remote pharmacy and/or from another expertise node such a doctor or nurse station, either by a dedicated link or through the Internet. Updating includes primary updating being the establishing for a period of time of the schedule of medication elements and the associated compliance monitoring. Updating can also include the downloading of applications related either directly to operation of the patient unit or using the patient unit as a storage, computing and network device. Such applications can include, for example, video conferencing, aspects of health monitoring, diagnostic support, etc.

Wide distribution of the patient unit of the invention to large groups of patients presents significant added value in terms of gathering and integrating medication and health records. Hospital records are generally focused on relatively severe, short term situations. The records that can be gathered and mined using networked patient units 29 of the type described, once a high level of market penetration has been reached, are somewhat different. Thus, they can present over the long term statistical data gathered from a large community and related to such issues as compliance, condition deterioration and improvement dynamics, responsiveness to specific drugs, etc. As shown in FIG. 16, such information may be analyzed, modified and made available from the central server 75 through any of a number of portals connected to the server, specific presentations being accessible only by respective users and the information presented being tailored to the particular needs of the respective users.

A central container packing and distribution facility such as a pharmacy 79 has access to the prescribed course of drugs being taken by a patient and an alert is sent over the network to the pharmacy when the cartridge is nearing exhaustion. At this point a replacement cartridge is packed at the pharmacy and, at the time that the replacement cartridge is sent out to the patient, a signal is sent to the patient or the patient's care giver informing them that a replacement cartridge is in transit to them. Re-fill of medication is relatively straightforward. The replacement cartridge is prepacked at the pharmacy or at a cartridge preparation and dispatch facility 77 according to the required prescription and schedule and is then sealed for transport to the patient unit site. The replacement cartridge is dispatched by mail or courier to the patient or care giver at the patient unit before full exhaustion of the original cartridge. Alternatively, a replacement cartridge can be picked up from a pharmacy following arrangements to have the cartridge sent from a packaging site. Upon receipt, the patient or local care giver opens the patient unit housing, extracts the spent cartridge, unseals the new cartridge, mounts the new cartridge in the housing with any required engagement with the feed mechanism, and returns the spent cartridge to the pharmacy for restocking. As shown in FIG. 16, replacement medication is dispensed under the direction of a pharmacist at a pharmacy 79 with cartridge preparation occur at a cartridge preparation and dispatch facility 77.

For the packing of a cartridge of the sort illustrated in FIG. 1, as shown in FIG. 17, the containers are formed from a web 17 of packaging material such as polyethylene. Prescribed medication elements such as preset quantities of pills 14 are accurately positioned at storage sites on the web 17, which is intermittently driven past a container manufacturing station (not shown). At this station, the web 17 is folded around the previously positioned medication elements 14 and heat sealed as shown at 18 around the elements at the web free edges, at the fold, and at the site of the separation zones 20. The web 17 is then advanced one container length to present the next part of the web to receive a further medication element and to be formed as the next container. Once the correct sequence of medication elements have been packed sufficient for the prescribed course of treatment and for the desired scheduled length of the course, the web 17 is cut and medication elements for another prescribed course of treatment are inserted in a further sequence of container filling and forming steps. For liquid medication, a preformed sachet filled with the required amount of the medication is dropped onto the web material and then made up into a medicine container as previously described with respect to pills.

The stored data related to the prescribed course of treatment to be delivered together with the record of drugs that have been successfully administered is maintained at the pharmacy centre and, as well as being used for medication verification, may be made available to other nodes on the network such as to a doctor or hospital site so as to present, over time, a complete patient medication history. The stored data may also be applied to the cartridge itself to enable inspection of the scheduled course of treatment locally at the patient unit 29.

As shown in FIG. 16, a variation of the patient unit of FIG. 8 includes a primary care test unit 71 for subjecting a patient to a test related to a property of the patient's condition. The test unit, measures the property to derive an indication of whether the patient has consumed the medication element. The primary care test unit 71 can be coupled into the patient unit so as to form part of the drug compliance system. In operation, in order for the patient to be fully compliant, he or she subjects him- or herself to a test for monitoring a physical change expected from consuming the scheduled medication. If the patient fails to undergo the physical test or if, upon undergoing the test, the test unit does not detect the expected physical change, an alert is issued over the network that non-compliance is suspected. The primary care test depends on the nature of the expected physical change which depends, in turn, on the drug being taken. The test may be a two-part test including measuring a patient condition before the delivered drug is consumed and then measuring the change in patient condition after the delivered drug has been taken. The patient unit can be operable to issue both a primary alert corresponding to an apparent failure by the patient or care giver to issue notification that a scheduled medication has been consumed and a secondary alert corresponding to the absence of an expected physical change following a scheduled consuming of medication. This aspect is of value for patients who may be particularly forgetful or who may have become distracted during the drug consumption procedure.

It is recognized, for example, that the incidence of medication treatment non-compliance for type 2 diabetes is relatively high. For assessing compliance with a diabetes medication course of treatment, a blood glucose meter is connected to the medication delivery storage and delivery unit. At each alert informing the patient that it is time to take the scheduled medication element, and immediately before the medication element is taken, the patient is requested to undergo a test to measure blood sugar level. In one example, a diabetes sufferer should, pursuant to a prescribed course of treatment, take a diabetes medication such as metformin a half hour before each meal to control blood sugar level after eating. With regard to the patient's mid-day meal, the patient may be required by the schedule to take the metformin medication at 11:30 a.m. The patient as part of the 11:30 a.m. process has blood glucose level measured at a measurement unit coupled to the medication delivery apparatus with a result, for example, that the blood glucose level is found to be in a normal range. The patient consumes the scheduled dosage of metformin and consumes his or her midday meal. Some time after the meal, between 2 to 2:30 pm, the patient's blood glucose is again measured at the measurement unit, with a measurement of 9 mmol being detected. This elevated blood glucose level is normal after a meal. However, had the patient missed the 11:30 a.m. dosage of metformin, s/he may have a measured blood glucose level of 14 mmol, for example, which would strongly suggest that the 11:30 am metformin consumption was missed.

In another example, over several days, a patient's blood glucose measured trend may indicate a blood sugar level that decreases to a normal range indicating that the patient's diabetes medication regimen is working. Data related both to medication compliance and the patient's blood glucose is transmitted onto the network and considered by a medical professional for the latter's evaluation as to whether taking a course the medication is proving to be effective for the patient, and, if not, whether the nature or strength of the prescribed medication needs to be changed.

A further example relates to a patient suffering from high blood pressure who is required periodically to take a vasodilator such as amlodipine besylate, The patient takes the amlodipine besylate at noon every day after measures his/her blood pressure reading at a blood pressure measuring unit coupled to the medication delivery apparatus. The patient's goal may be to manage systolic over diastolic readings to be below 130 over 80, so that 140 over 100 would be indicative of hypertension. If the patient misses consumption on day 1, the patient's day 2 reading might typically show slight elevation (125 over 85). If the patient also misses consumption on day 2, the elevation can be expected to be more pronounced on day 3; for example, 128 over 90. This will suggest to the person on the network responsible for monitoring the patient's compliance that the patient is not taking the anti-hypertensive medication.

A final example relates to alleviating chronic pain. At the time of an alert indicating to the patient that medication should be taken, the patient is requested to complete a pain score survey on a device having an interface to the patient unit. For example, the patient may report a “7” (where 0 is no pain and 10 is a highest level of pain) and then consumes the presented medication element and confirms consumption at the compliance unit. Some period of time later, either prior to or at next consumption reminder, the patient again completes the survey and, at this time, may report, for example, a pain level of “5”. A medically trained professional considers the patient's pain sensation data in conjunction with the compliance report to evaluate whether the medication is proving to be effective and whether the prescribed strength requires adjustment. It will be appreciated that this test is a subjective test as opposed to the objective tests of the prior examples.

As shown in FIG. 16, the network optionally includes a vital statistics test unit 72 having functionality similar to the primary care test unit 71 previously described, but adapted to be used for several tests and without being part of a medication compliance system. Such a test unit is used to monitor aspects of a patient's health condition, irrespective of the fact or timing of any medication treatments. Thus, on an ad hoc or scheduled basis, the vital statistics test unit is caused to signal to the patient that he or she is to submit to one or more tests, the results of which are input to the system and analyzed at a remote location to assess some aspect(s) of the patient's health and, in particular, to detect any deterioration in condition. The vital statistics test unit may include any of a battery of standard test units.

These preferably include non-invasive diagnostic elements. For measuring blood pressure, such apparatus can include, for example, Doppler blood-pressure monitoring devices or automated oscillometric monitoring devices. For measuring temperature, such apparatus can include, for example, non-contact, infra red reading thermometers. For measuring weight, such apparatus can include a load-cell scale incorporated in a mat on which the patient will stand. Such apparatus can further include finger pulse oximeters for measuring blood oxygen levels and electronic stethoscopes for monitoring respiratory condition. Such apparatus can include breath sampling and breath analyzer units particularly for evaluating outgas products such as alcohols or ketones. Such apparatus can further include pupil retina scanning devices for generating basic information related to possible stress, fatigue and brain functions. In each case, the monitoring or measuring units generate electrical outputs which are input to the patient unit and converted to signals indicative of patient condition. The patient vital statistics data is then transmitted onto the network for access by a service centre, the patient's doctor or nurse, or by his or her care giver. It will be understood that in addition to generally commercially available diagnostic elements, the particular devices, or a combination of them, can be tailored to the particular health concerns of a patient. It will be understood too that part at least of the monitoring and measuring apparatus, such as transducers and electronic processing hardware may be housed in the patient unit.

An alternative embodiment of the primary care test unit is used where a patient has a condition which varies in severity over time and where, in response, more or less of maintenance medication should be taken. A special cartridge is used for this condition, the cartridge having sub-elements of a required drug in adjacent containers. If the patient's condition improves such that a lower amount of the prescribed drug is needed, some of the sub-elements are sent to a discard station instead of being delivered, or the patient is signaled that he or she should not consume the medication in the next container to be dispensed. If, on the other hand, the patient's condition worsens and a higher amount of the medication is once again required, the patient is informed that medication contained in all of the delivered containers should be consumed.

The patient unit may also have an associated set of diagnostic functionalities with the communications link being used to effect either or both of prompting the patient to undergo a test such as for example, measurement of blood pressure or sugar level, and providing the results of the diagnostic procedure to a doctor or other medical expert or resource on the network. The diagnostic functionalities are either mounted within the patent unit or are mounted in a standalone unit which is networked with the patient unit to permit data to be made available to other nodes on the network.

It will be seen that the use of the cartridge and the patient unit provide patient safety in two respects. Firstly, if the medication delivery procedure is not followed within a required period of time, an alert is triggered and corrective action is initiated. Secondly, access to stored medication other than a dose scheduled for use is discouraged. In this respect, the cartridge does not lend itself to wrongful dose consumption since only the scheduled dose is presented at the exit slot and the cartridge itself is rolled up and mounted in the housing with later doses confined within the cartridge roll. This latter safety aspect can be supplemented by having the housing locked once the cartridge is loaded. Ideally, the consumption of the drug is made as simple as possible with the minimum amount of involvement of the patient in the selection of the drug to be taken.

It will be appreciated that while a cartridge of physically linked medication containers is a particularly convenient way of ordering medication elements for subsequent delivery to the patient, neither the cartridge form nor the physical linking of the medication containers is essential. Thus, for example, the individual containers are stored in a random way within a storage bay of the patient unit, with each container marked with a barcode signifying at least the scheduled time of consumption of the medication element contained in the respective container. At a treatment session, a corresponding delivery mechanism reads the codes on the containers to find the container next scheduled for delivery and brings that container to the inspection station. As in the previously described embodiment, information derived from the code on that next scheduled container is compared to the stored data to verify that the container contains the right medication element, that there has been no recall, that the medication element expiry date has not passed, etc.

Other variations and modifications will be apparent to those skilled in the art. The embodiments of the invention described and illustrated are not intended to be limiting. The principles of the invention contemplate many alternatives having advantages and properties evident in the exemplary embodiments.

What is claimed is:

1. A medication treatment compliance apparatus for use in a medication compliance system, the medication treatment compliance apparatus configurable as a node on a communication network and comprising: a storage unit for storing a plurality of medication elements together comprising a prescribed course of treatment;
a delivery unit for delivering the medication elements in a scheduled order corresponding to the course of treatment;
a notification unit for notifying a patient at the medication delivery apparatus that the patient should take a next scheduled one of the medication elements;
a compliance unit for issuing to the network an indication that the next scheduled one of the medication elements has been consumed by the patient.

2. A medication treatment compliance apparatus as claimed in claim 1, the apparatus forming one node on a communication network, the compliance unit operable to issue the notification to another node on the network for the attention of a compliance agent.

3. A medication treatment compliance apparatus as claimed in claim 1, wherein the patient actuates the compliance unit to issue said indication to the network.

4. A medication treatment compliance apparatus as claimed in claim 1, further comprising detection means to detect whether the patient has consumed the next scheduled one of the medication elements and automatically to issue the alert if it is detected that the patient has consumed the medication element.

5. A medication treatment compliance apparatus as claimed in claim 2, further including video conferencing elements for providing video conferencing between the patient unit and another node on the communication network.

6. A medication treatment compliance system including a compliance apparatus as claimed in claim 2, the system including the other node, a compliance agent at the other node to monitor the alert on one of a scheduled basis and an on-demand basis.

7. A medication treatment compliance apparatus as claimed in claim 2, further comprising a summons functionality for connection to the network to enable a person at the patient unit to summon assistance from the network.

8. A medication treatment compliance apparatus as claimed in claim 2, further comprising a summons functionality for connection to the network to enable a person at a remote node on the network to summon a person at the patient unit.

9. A medication treatment compliance system as claimed in claim 6, further comprising control hardware and software for controlling operation of the medication treatment compliance apparatus, at least a part of the control hardware and software located at a node remote from the medication treatment compliance apparatus.

10. A medication treatment compliance apparatus as claimed in claim 2, further comprising a pre-emptive functionality for prompting delivery from the delivery unit of a medication element at a time earlier than a scheduled time for a patient to consume the medication element if complying with the prescribed course of treatment to allow the patient to take the medication element to a location remote from the medication treatment compliance apparatus, the system further comprising a mobile device having an alert capability to alert a patient at the remote location to consume the medication element at the scheduled time for the patient to consume the medication element if complying with the prescribed course of treatment.

11. A medication treatment compliance apparatus as claimed in claim 9, the mobile device having a compliance function actuatable by the patient to send onto the network an indication that a scheduled medication element has been consumed.

12. A medication treatment compliance apparatus as claimed in claim 1 further comprising a primary care test unit for subjecting a patient to a test related to a property of the patient's condition, and for measuring the property to derive an indication of whether the patient has consumed the medication element.

13. A medication treatment compliance system as claimed in claim 11, the a primary care test unit operable to measuring the property following one of a single instance of scheduled consuming of a medication element and a period corresponding to multiple instances of scheduled consuming of medication elements.

14. A method of medication treatment compliance comprising, at a medication treatment compliance apparatus configured as a node on a communication network:
storing a plurality of medication elements in a storage unit, the medication elements together comprising a course of treatment;
configuring a delivery unit for delivering the medication elements in a scheduled order corresponding to the course of treatment;
operating a notification unit for notifying a patient at the medication delivery apparatus that the patient should take a next scheduled one of the medication elements;
issuing to the network an indication that the next scheduled one of the medication elements has been consumed by the patient.

15. A method as claimed in claim 14, the storage unit and delivery units together forming a patient unit, the patient unit forming one node on communication network, the method further comprising the notification being issued from said one communication node to another node on the network, and the alert being issued from said one communication node to another node on the network.

16. A method as claimed in claim 15, further comprising establishing video conferencing between the patient unit and the other node on the communication network to investigate the issuing of the alert.

17. A method as claimed in claim 15, further comprising a remote contact person monitoring the alert at the other network node on one of a scheduled basis and an on-demand basis.

18. A method as claimed in claim 15, further comprising issuing from the patient unit onto the network a notification that a medication element scheduled to be consumed has been consumed.

19. A method as claimed in claim 15, the patient unit further comprising operating a summons functionality connected to the network to enable a person at the patient unit to summon assistance from the network.

20. A method as claimed in claim 15, the patient unit further comprising operating a summons functionality connected to the network to enable a person at a remote node on the network to summon a person at the patient unit.

21. A method as claimed in claim 15, operating a pre-emptive functionality at the patient unit to deliver from the delivery unit a container containing a medication element at a time earlier than a scheduled time for a patient to consume the medication element if complying with the prescribed course of treatment, the patient taking the pre-emptively delivered medication element to a remote location, and receiving an alert from the network on a mobile device to alert a patient at the remote location to consume the pre-emptively delivered medication element at said scheduled time.

22. A method as claimed in claim 15, further comprising conducting a test on the patient to measure a property of the patient's condition, comparing whether the measure of the property is consistent with the patient having consumed the medication element, and issuing said alert if the measure of the property is not consistent with the patient having consumed the medication element.

23. A method as claimed in claim 22, further comprising measuring the property following one of a single instance of a scheduled consuming of a medication element and a period for a scheduled multiple consuming of medication elements.

24. A method as claimed in claim 22, further comprising measuring the property within a predetermined period after the medication element is schedule to be consumed.

25. A method as claimed in claim 22, further comprising measuring the property before a scheduled consuming of a medication element, measuring the property after the scheduled consuming of the medication element, by comparing the two measurements computing a change in the property, and issuing the alert if the change in the property is indicative of the scheduled medication element not having been consumed.

26. A method as claimed in claim 15, further comprising escalating a level of the issued alert in response to communication between the patient unit and another node on the network showing that a medical element scheduled to be consumed still has not been consumed by a predetermined period after the issuing of the alert.

27. A method as claimed in claim in claim 26 in which the escalating of the level of the issued alert involves the establishment of communication on the network with at least one of a pharmacist, a doctor, and emergency services.
